(19) United States
(12) Patent Application Publication
(10) Pub. No.: US 2016/0216252 A1
(43) Pub. Date: Jul. 28, 2016

(54) PLASMONIC BEADS FOR MULTIPLEXED ANALYSIS BY FLOW DETECTION SYSTEMS

(71) Applicant: The Board of Trustees of the Leland Stanford Junior University, Stanford, CA (US)

(72) Inventors: Bo Zhang, Stanford, CA (US); Hongjie Dai, Cupertino, CA (US); Yingping Zou, Shanghai (CN); Ming Gong, Stanford, CA (US); Jiang Yang, Mountain View, CA (US)

(21) Appl. No.: 14/917,525

(22) PCT Filed: Sep. 11, 2014
(86) PCT No.: PCT/US2014/055228

§ 371 Date: Mar. 8, 2016

Related U.S. Application Data
(60) Provisional application No. 61/877,782, filed on Sep. 13, 2013.

(51) Int. Cl. G01N 33/533; G01N 33/553; G01N 33/542

(52) U.S. Cl. CPC G01N 33/533; G01N 33/542; G01N 33/553

(57) ABSTRACT

Disclosed are methods and assays for detection of low concentration analytes such as proteins in a sample, using beads. Specially coated beads allow for femtomolar sensitivity through strong near-infrared fluorescence enhancement on plasmonic beads having gold nanostructures in a coating. By selecting different bead sizes and labeling with different fluorophores of plasmonic beads for immobilization of different capture antibodies, multiplexed plasmonic beads can be used for simultaneous quantification of various markers down to 0.01 pg/mL sensitivity. Exemplified are human cytokine IL-6, IFN-gamma, IL-1 beta, VEGF and ovarian cancer biomarker CA-125. Using flow cytometry, a detection limit below that of glass bead based immunoassays by 2-3 orders of magnitude was achieved. The multiplexed plasmonic bead assay was used to simultaneously quantify cytokines and CA125 of ovarian cancer cell culture medium, demonstrating the potential of plasmonic bead based immunoassay for sensitive biological detection relevant to human diseases.

### CROSS-REFERENCE TO RELATED APPLICATIONS

This application claims priority from U.S. Provisional Patent Application No. 61/877,782 filed on Sep. 13, 2013, which is hereby incorporated by reference in its entirety.

### STATEMENT OF GOVERNMENTAL SUPPORT

This invention was made with Government support under contract 5R01CA135109-02 awarded by the National Institutes of Health. The Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the fields of analyte detection, microparticles or beads in suspension, flow cytometry, surface plasmon resonance and metal-enhanced fluorescence, and multiplexed immunoassays for detecting ultra-low (i.e. sub-pg/mL) amounts of (for example) protein biomarkers.

2. Related Art

Presented below is background information on certain aspects of the present invention as they may relate to technical features referred to in the detailed description, but not necessarily described in detail. The discussion below should not be construed as an admission as to the relevance of the information to the claimed invention or the prior art effect of the material described.

Since the initial introduction in 1977¹, bead based flow cytometric immunoassays have become widely explored for quantitative protein analysis. Among various types of protein detection platforms, a bead based assay is favored for reduced assay processing time through combining rapid solution-phase kinetics, the ability for multiplexed protein analysis, and the wide availability of flow cytometry². Accompanied by shortened assay process and low cost of samples, reagents and labor, bead based immunoassay analysis has been growingly favored for research and clinical use^{3,4}. Currently, commercially available bead based assays have been widely used for cytokine quantification^{5,6}; bead based flow cytometric technology has also been applied for biomarker searching⁷ and microRNA expression profiling for cancer study⁸.

Typical bead based flow cytometric assays afford a low limit of cytokine detection of ˜1 pM level (1-10 pg/ml)^{9,10}, spanning ˜4 orders of dynamic range. Cytokines are important in regulating hematopoiesis, immune response, cellular activity and are typically expressed at low levels between sub-pg/ml to thousands of pg/ml¹¹. Cytokines at expression levels lower than 1 pg/ml are below the detection limit of bead based immunoassay and the biological function of cytokines at such levels and correlation with diseases states are difficult to assess and remain unclear^{12,13}. Innovations in bead based flow cytometric assay with improved sensitivity will enable more accurate protein analysis at sub pg/ml level, as highly desired in the cytokine case.

Planar gold plasmonic films have been recently investigated and applied for sensitive biomolecular analysis in planar protein and antibody microarrays, utilizing plasmonic near-infrared fluorescence enhancement (NIR-FE)^{15,16}. Although silver nanoparticles have been applied to silica spheres for fluorescence enhancement of ˜10 times in the visible¹⁷, multiplexed plasmonic beads for flow cytometric immunoassays have not been previously achieved. Also, the 10-fold fluorescence enhancement afforded by Ag coated beads is much smaller than the enhancement achieved here, and Ag enhancement is only achieved in the visible emission range which suffers from higher noise levels due to higher autofluorescence background in this range. As a result, bio-assay sensitivity afforded by silver coated beads is similar to non-metal coated beads. Further, Ag is unstable and oxidizes in air to lose its enhancement ability. As a result, Ag coated beads have not been adopted by the immunoassay field. Surface plasmons on metal surfaces have been found to be able to couple to fluorophores at excited-state, increasing the radiative decay rate of the excited fluorophores, thus enhancing fluorescence quantum yield in microwave accelerated metal enhanced fluorescence, using silver¹⁴.

### SPECIFIC PATENTS AND PUBLICATIONS

Tabakman, S. M. et al. “Plasmonic substrates for multiplexed protein microarrays with femtomolar sensitivity and broad dynamic range,” Nature Communications 2, 466, doi:10.1038/ncomms1477 (2011) (Ref. 15) discloses a nanostructured, plasmonic gold film on a “protein chip” microarray. The authors demonstrate a multiplexed autoantigen array for human autoantibodies implicated in a range of autoimmune diseases.
US 2013/0172207 “Fluorescence enhancing nanoscopic gold films and assays based thereon,” published Jul. 4, 2013 by inventors including those listed here and assigned to the same assignee is related to the above publication. This disclosure concerns NIR-FE based biological detection in microarray format on planar substrates,
Deng et al., “Enhanced Flow Cytometry-Based Bead Immunoassays Using Metal Nanostructures,” Analytical chemistry 81, 7248-7255 (2009) (Ref. 17) discloses metal-enhanced fluorescence emission of fluorophores located on the surface of silica beads coated with nanostructured silver, suitable for flow cytometry detection. The fluorescence enhancement was investigated using a model AlexaFluor 430 IgG immunoassay and AlexaFluor 430 labeling. Approximately 8.5-fold and 10.1-fold higher fluorescence intensities at 430 nm excitation were, respectively, observed from silvered 400 nm and 5 μm silica beads deposited on glass as compared to the control sample.

### BRIEF SUMMARY OF THE INVENTION

In certain aspects, the present invention comprises methods and materials in which fluorophore conjugated detection molecules are measured on individual beads (also termed “microparticles”) for signal reporting in a bead based flow cytometric assay. The invention comprises strategies for amplifying fluorescent emission of fluorophores on each bead by >10-50 fold, of fluorescence emission or >50 fold, that will lead to much improved sensitivity for analysis of low concentration analytes such as proteins. The present invention comprises, in certain aspects, a method for detecting analytes in a sample, comprising a step of contacting the sample with a population of microparticles in suspension, where said microparticles comprise a plasmonically active surface providing red and near-infrared (“NIR” as described below) enhanced fluorescence in the region of 500-1700 nm, preferably in the range of 650-1700 nm emission, for use with various fluorophores including organic dyes, polymers and inorganic nanoparticles/nanocrystals. The population of microparticles may be provided with the NIR enhanced fluorescence by being completely or substantially coated with gold nano-islands, as illustrated in FIG. 1A. The population of NIR enhanced microparticles further comprises subpopulations of different analyte capture molecules bound to said plasmonically active surface on various microparticles in the population. The analyte capture molecule on a given subpopulation is tailored to the analyte to be detected. That is, the analyte capture molecule may be an antibody to an antigen analyte, or, conversely, it could be an antigen ligand to an antibody analyte. It could also be, e.g. an oligonucleotide complementary to a nucleic acid analyte.

The method further comprises preparing different analyte capture molecules on different microparticles in suspension, and allowing them to form complexes with different analytes that may be present in the sample; labeling said complexes with fluorescent labels; and detecting labeled complexes by irradiating said fluorescent labels and sensing metal enhanced fluorescence from labeled complexes, whereby said enhanced NIR fluorescence indicates the presence of one or more analytes. Multiple analytes in the sample may be distinguished by different enhanced NIR fluorescence signals. The step of distinguishing multiple analytes comprises use of one or more of (i) different fluorescent labels attached to different captured analytes, such as where a secondary antibody or streptavidin-fluorophore is used to bind analyte captured by a capture antibody on a bead, or an antibody captured by an antigen on a microparticle, i.e. bead; (ii) differences in microparticle sizes used to detect different analytes, since it has been found that bead size affects the scattering signals generated in a flow cytometer; or (iii) different labels affixed to different microparticles used to detect different analytes. The different labels may be absent or present in or on the bead, and are present whether or not analyte is bound.

The present invention comprises, in certain aspects, a method wherein the step of detecting and distinguishing one from the other multiple analytes in a sample, where the method comprises measuring different enhanced NIR fluorescent signals by flow cytometry. The present invention comprises, in certain aspects, use of fluorescent labels that are NIR dyes, e.g. cyanine dye, Alexa dyes, IR dyes, CF dyes, Atto dyes, Dylight dyes, quantum dots, conjugated polymer dyes, and carbon nanotubes. An example of NIR dye used here is Cy5, where which Cy5 conjugates are excited maximally at 650 nm and fluoresce maximally at 670 nm. They can be excited to about 98% of maximum with a krypton/argon laser (647 nm line) or to about 63% of maximum with a helium/neon laser (633 nm line). A significant advantage of using Cy5 and DyLight 649 over other fluorophores is the low autofluorescence of biological specimens in this region of the spectrum. However, because of their emission maximum at 670 nm, they cannot be seen well by eye, and they cannot be excited optimally with a mercury lamp. Therefore, they are not recommended for use with conventional epifluorescence microscopes. They are most commonly visualized with a confocal microscope equipped with an appropriate laser for excitation and a far-red detector. Cy5 and DyLight 649 conjugates are a less expensive and equally bright alternative to Allophycocyanin conjugates for flow cytometry.

The present invention comprises, in certain aspects, methods wherein the plasmonically active surface on the microparticle comprises gold nano-islands. Gold nano-islands may be attached to an amine coating applied to silica-based, polymer-based or magnetic beads. The present invention comprises, in certain aspects, use of microparticles with an avidin layer or carboxylic acid or amine groups or thiol molecules on top of the plasmonically active surface.

The present invention comprises, in certain aspects, use of analyte capture molecules that are antibodies, antigens, carbohydrates, nucleic acids and binding peptides. The present invention comprises, in certain aspects, analyte capture molecules that are biotinylated antibodies bound to an avidin layer on a microparticle.

The present invention comprises, in certain aspects, methods and plasmonically active beads as described above wherein the step of labeling complexes with fluorescent labels comprises steps of providing said analyte capture molecules in the form of antibodies immobilized on the surfaces of microparticles; and providing a fluorescent label in the form of fluorescently labeled antibodies specific to said analytes, whereby a complex is formed between capture molecules, analyte, and fluorescently labeled antibodies. In certain embodiments, the step of distinguishing multiple analytes is done with antibodies having different specificities and labeled with different NIR labels having non-overlapping emission spectra.

The present invention comprises, in certain aspects, a product comprising a population of beads as described above wherein each bead comprises gold islands separated by gaps of between 5 and 100 nm, or between 10 and 100 nm, and the islands are between 1,000 and 2,500 nm², or between 25 and 250,000 nm², in area, said population of beads further having coupled thereto analyte capture molecules of at least two different specificities. This is considered dividing the population of beads into subpopulations, where each subpopulation comprises beads for a certain analyte. Multiplexing of subpopulations may comprise a large number of different specificities; six different specificities are exemplified in a multiplexing experiment. The different sizes of beads producing different signals may differ in diameter by at least a factor of 1.5. The beads may comprise different fluorescent labels attached in or on them. Bead size range is between 100 nm and 100,000 nm.

The present invention comprises, in certain aspects, a product as described above, wherein said analyte capture molecules are antibody molecules of at least two different specificities. The analyte capture molecules may be selected from the group consisting of antibodies, carbohydrates, and binding peptides.

The present invention comprises, in certain aspects, an immunoassay using an immunoassay product as defined above, wherein the assay detects analytes in the subpicomolar range and comprises the steps of:

(a) forming a mixture of a sample with said population of beads;

(b) allowing capture antibody or antigen or DNA or peptide molecules to bind to analytes in said sample;

(c) separating beads with bound analytes;

(d) forming a complex of bound analytes and fluorescently labeled detection molecules; and

(e) detecting the complex of step (d) by fluorescence, wherein said fluorescence is enhanced NIR fluorescence resulting from interaction between fluorescent labels on the fluorescently labeled detection molecules and the plasmonically active layer.

In certain aspects, the immunoassay may comprise detecting of a bound analyte by flow cytometry. In certain aspects, said flow cytometry may distinguish between different analytes by measuring both scattering and fluorescent emission peaks at different wavelengths. In certain aspects, the immunoassay may comprise detecting of a bound analyte by microscope imaging. In certain aspects, said microscope imaging may detect fluorescence in the visible infrared range, the NIR-I range, or the NIR-II range of the electromagnetic spectrum.

In certain aspects, the present invention comprises a method of making a bead-based immunoassay product comprising a population of beads, comprising the steps of: modifying beads of a silica or polymer or magnetic material with an amine functionality; coupling the modified beads to a plasmonically active layer comprising gold islands separated by gaps of between 5 and 100 nm, or between 10 and 100 nm, and between 1,000 and 2,500 nm², or between 25 and 250,000 nm², in island area; applying to beads a functionality for coupling thereto a population of detection molecules; and coupling detection molecules of different specificities to the beads.

### DESCRIPTION OF THE DRAWINGS

FIG. 1A is a schematic diagram showing a plasmonic bead according to the present invention coated with capture antibodies, with the target antigen, the detection antibody, and the attached label all complexed with the bead.

FIG. 1B-1D is set of images showing the nano-engineered plasmonic gold island pattern covering the glass bead. FIG. 1B shows a group of 8 micron glass beads uniformly covered with gold island structure (left) and a zoomed in region of the 8 micron bead demonstrating details of the gold island pattern on the bead (right). FIG. 1C shows a group of 4 micron glass beads uniformly covered with gold island structure (left) and a zoomed in region of one 4 micron bead demonstrating details of the gold island pattern on the bead (right). FIG. 1D is a schematic drawing of flow cytometry based fluorescence measurement, demonstrating the identification of a single bead from front scattering and side scattering of incident light, and the fluorescence quantification of the bead for a certain fluorophore.

FIG. 1E-1F is a pair of graphs showing Cy5 fluorescence measurement of Cy5-avidin coated plasmonic and glass beads by flow cytometry, demonstrating distribution of Cy5 fluorescence intensity on 5000 plasmonic beads and 5000 glass beads, respectively (FIG. 1E) and a mean Cy5 fluorescence intensity plot of the 5000 plasmonic beads and 5000 glass beads, showing >100 fold fluorescence enhancement on plasmonic beads (FIG. 1F).

FIG. 2A-2C is a series of images showing protein quantification on plasmonic beads. FIG. 2A is a schematic showing the sandwich structure for protein detection on a plasmonic Au bead and glass bead. FIG. 2B-2C is a pair of graphs showing human cytokine IL-6 calibration curves on a plasmonic bead (left) and glass bead (right), showing Cy5 fluorescence distribution when measuring: 1 nM IL-6, 100 pM IL-6, 10 pM IL-6, 1 pM IL-6, 100 fM IL-6, 10 fM IL-6 and a blank control.

FIG. 2D-2E is a pair of graphs showing Cy5 fluorescence quantification curves for IL-6 detection on plasmonic beads (FIG. 2D) and glass beads (FIG. 2E).

FIG. 3A is a schematic drawing showing the design for the 8 micron and 4 micron plasmonic bead systems for 6-plexed biomarker measurement.

FIG. 3B-3C is a series of flow cytometry scatter plots showing the 3-dimensional bead system. 4 micron and 8 micron beads are resolved from the side-scattering versus front-scattering plot (FIG. 3B, top left). In each bead sized region, 3 sub-regions can be resolved from the Cy3 fluorescence versus Alexa fluor 488 fluorescence plots, which are Cy3 coded (labeled) beads, Alexa 488 coded beads, and non-coding beads. FIG. 3C is an image showing confocal fluorescence mapping of the original 6-plexed bead system before biomarker sensing: Cy3 fluorescence (2, 5); Alexa 488 fluorescence (3, 6).

FIG. 4A is a pair of images showing confocal fluorescence mapping of the 6-plexed bead system before and after biomarker sensing of a mixture of CA-125, IFN-gamma, IL-6, VEGF, IL-1 beta each at 100 pM.

FIG. 4B, 4C, 4D is a set of graphs showing fluorescence quantification curves for serial dilutions of CA-125 antigen, cytokine IL-6 and IL-1 beta.

FIG. 4E, 4F, 4G is a set of bar graphs showing selectivity tests of the multiplexed plasmonic bead assay, reflecting fluorescence of each of the 6 sub-region of plasmonic beads where 10 U/ml CA-125, 1 pM IL-6 or 1 pM IL-1 beta was applied for biomarker quantification with the multiplexed plasmonic beads system.

FIG. 5A is a schematic drawing of a 6-plex bead solution for cell culture medium sensing after 48 h of cell culture and several cytokines+CA 125 were picked up from the medium by flow cytometry measurement.

FIG. 5B-5E is a series of graphs showing biomarker sensing results for ovarian cancer cell line OVCAR3 (FIG. 5B) and SKOV3 (FIG. 5C) culture mediums and the quantification of protein biomarker concentration of OVCAR3 (FIG. 5D) and SKOV3 (FIG. 5E) cell lines by fitting Cy5 mean fluorescence into the calibration curve of each biomarker.

FIG. 6A, 6B is a series of images and a bar graph showing fluorescent enhancement of the gold coated plasmonic beads at growth concentrations of 45 μM, 75 μM, 95 μM, 110 μM, 120 μM, 145 μM, and 160 μM.

FIG. 7A, 7B is a series of images and a bar graph showing fluorescent enhancement of the gold coated plasmonic beads at growth concentrations of 45 μM, 75 μM, 80 μM, 87.5 μM, 95 μM, 110 μM, and 125 μM.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although methods and materials similar or equivalent to those described herein can be used in the practice or testing of the present invention, the preferred methods and materials are described. Generally, nomenclatures utilized in connection with, and techniques of, physics, biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. A subrange is to be included within a range even though no sub-range is explicitly stated in connection with the range. As a nonlimiting example, a range of 120 to 250 includes a range of 120-121, 120-130, 200-225, 121-250 etc. The term “about” has its ordinary meaning of approximately and may be determined in context by experimental variability. In case of doubt, “about” means plus or minus 5% of a stated numerical value. The term “each” is used herein in a denominative sense to address individual objects composing a number of objects, considered separately from the rest, and does not imply “each and every” object referred to.

The term “protein” means a polymer of amino acids without regard to the length of the polymer, provided that the protein has specific binding properties. The proteins include but are not limited to cytokines, cancer biomarker proteins, antibodies and antigens related to infectious and autoimmune diseases, and protein and antibody biomarkers for cardiovascular diseases. This term also does not specify or exclude chemical or post-expression modifications of the polypeptides of the invention, although chemical or post-expression modifications of these polypeptides may be included or excluded as specific embodiments. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12, 1983; Seifter et al., Meth Enzymol 182:626-646, 1990; Rattan et al., Ann NY Acad Sci 663:48-62, 1992). Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

Many proteins are antigens known for use in immunoassays. For example, CEA is a glycoprotein involved in cell adhesion and has been implicated as a cancer biomarker. Included specifically within this definition and contemplated for use herein are human serum proteins and human intracellular proteins.

The term “analyte capture molecule” refers here to molecules that specifically bind to an analyte of interest with sufficient avidity to allow the analyte to remain attached to the analyte capture molecule during a detection process. The specificity, as is commonly understood, refers to the ability of the analyte capture molecule to discriminate between related analytes, and bind only to the analyte of interest. The required degree of specificity will depend on the application; for example, an analyte capture molecule for detecting VEGF may be suitable if it binds to all four isotypes of VEGF. Suitable analyte capture molecules may be a nucleic acid, a receptor, an enzyme, an antibody or an antibody-like molecule, a protein, amino acids etc. In certain applications, an analyte capture molecule may be a molecular complex, including an entire cell, virion, or organelle.

The term “antibody” means any of several classes of structurally related proteins, also known as immunoglobulins, that function as part of the immune response of an animal, which proteins include IgG, IgD, IgE, IgA, IgM and related proteins which specifically bind to their cognate antigens. The term “antibody” here refers to an antibody specifically binding to a single antigen specificity rather than a mixed population of antibodies. Antibodies as contemplated herein are any antibody-like molecule useful in an immunoassay, including known direct and indirect (“sandwich”) immunoassays.

The term “specific binding” means that binding which occurs between such paired species as enzyme/substrate, receptor/agonist or antagonist, antibody/antigen, complementary polynucleotides (polynucleic acids) and lectin/carbohydrate which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specifically binding” means binding between a paired species where there is interaction between the two, which produces a bound complex having the characteristics of an antibody/antigen or enzyme/substrate interaction. In particular, the specific binding is characterized by the binding of one member of a pair to a particular species and to no other species within the family of compounds to which the corresponding binding member belongs. Thus, for example, an antibody preferably binds to a single epitope and to no other epitope within the family of proteins. This is distinguished from non-specific binding, such as nonspecific binding to surfaces like ELISA wells or Western blotting membranes, or binding to substances in solution which are present in high concentration e.g. albumin or immunoglobulin.

In certain embodiments, molecules for specific binding are antibodies, carbohydrates, and binding peptides. They may be combined on a single bead. Antibodies are discussed below. Carbohydrates may be used to specifically recognize as analytes lectins. Lectins may be present on mammalian tumor cells. Carbohydrates attached to glycoproteins may also be used on the present beads. The term “binding peptide” refers to a non-antibody polypeptide that can specifically recognize an analyte in a sample. A binding peptide may be, e.g. a receptor or a fragment of a natural ligand for a receptor. Peptides that bind to receptors are described, e.g. in US 20130079292, US 20130130979, and US 20120270238. It could be a covalent probe binding to an enzyme.

The term “polynucleotide” or “polynucleic acid” means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g., naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Polynucleotides typically range in size from a few monomeric units, e.g., 5-40, when they are usually referred to as “oligonucleotides,” to several thousand monomeric units. Polynucleotides are contemplated as analytes for detection in the present assays, and may be also used as part of a labeling step, through specific hybridization. As discussed below, polynucleotides or oligonucleotides may be immobilized on a microparticle and used for detecting complementary nucleic acids in a sample.

The term “plasmonically active” is used in reference to a material that supports electronic plasmons, particularly surface plasmons, thereby exhibiting plasmonic properties. Surface plasmons have been used to enhance the surface sensitivity of several spectroscopic measurements including fluorescence, Raman scattering, and second harmonic generation. The term may be more fully understood by reference to Wilson et al. “Directly fabricated nanoparticles for Raman scattering,” US Pub. 20110250464. The phrase “plasmonic properties” refers to properties exhibited by surface plasmons, or the collective oscillations of electrical charge on the surfaces of metals. In this sense, plasmonic properties refers to measurable properties, as described e.g. in Nagao et al. “Plasmons in nanoscale and atomic-scale systems,” Sci. Technol. Adv. Mater. 11 (2010) 054506 (12 pp), describing plasmonic sensors, such as those used for surface-enhanced IR absorption spectroscopy (SEIRA), surface-enhanced Raman scattering (SERS). Another plasmonic property is plasmon-enhanced fluorescence, described e.g. in Sensors and Actuators B 107 (2005) 148-153. That study presented a nanosphere lithography technique to produce silver nanostructures to enhance fluorescent dye Cy5 by ˜10 times, although without demonstrating any improvement in bioassays.

“Gold nano-islands” are described in detail in the inventor's co-pending application entitled “Fluorescence enhancing nanoscopic gold films and assays based thereon”, Ser. No. 13/728,798, US 2013/0172207, published Jul. 4, 2013. Briefly, as described there, the method comprises a three step process in the preparation of the present nanoscopic (“Au/Au”) films:

(1) seeding of gold onto a micro-bead by precipitation out of a solution of Au³⁺ ions. The ions are precipitated from HAuCl₄ by raising its pH with a nitrogenous base, such as with NH₄OH, urea, etc;
(2) reducing the ions precipitated in step (1) to Au₀ clusters on the a micro-bead by a reducing agent such as NaBH₄, heat or H₂; and
(3) growing seeds from step (2) by selectively adding gold to the initial seeds by reduction of an Au³⁺ halide in a second solution to form “islands.” This can be done by a reducing agent such as hydroxylamine. The additional gold in step (3) only attaches to the previously deposited seeds, leading to the present so-called “Au/Au” or gold-on-gold construction. The method produces random gold nano-islands at ˜5-100 nm, or ˜10-100 nm, nano-gap spacing on a micro-bead. Following reduction of the seeds, one will obtain nanoislands with heights 5-200 nm, or 5-500 nm.

The term “metal enhanced fluorescence” or “MEF” is used in its commonly accepted sense of an enhancement of fluorescent intensity of a fluorophore in proximity to a metal where fluorophores in the excited state undergo resonance interactions with the surface plasmons in the metal particles. The enhancement results from the plasmon-coupling and electric field amplification.

The term “sample” is used in a broad sense to include any material, including an organic material, living, or non-living, that may exist in nature, or be created by a natural process. A sample may be synthetic, e.g. when one wishes to measure the amount of or presence of an inorganic substance in a mineral sample. The sample will be presumed to contain an analyte, that is, the chemical or biological substance that undergoes analysis or detection in an assay.

The term “NIR” as used herein, means red and near infra-red, specifically in the range of 650-1700 nm. The term covers the red and the near infra-red region of the electromagnetic spectrum (from 0.75 to 3 μm). For purposes of biological imaging, the present NIR range is divided into visible NIR, around 650-750 μm, NIR-I, around 800 nm (0.75-0.9 μm) and NIR-II, between about 1 (e.g. 1.1 μm) to 1.7 μm for fluorophores like carbon nanotubes, certain conjugated polymers, small molecules and certain quantum dots.

The term “NIR label” means fluorophores such as organic molecules, polymers, carbon nanotubes and quantum dots emitting in the NIR region of 650-1700 nm range (as defined above). Examples of NIR labels are carbocyanine dye (for example, an indocyanine dye), that optionally comprises a functional group, for example, a succinimidyl ester, that facilitates covalent linkage to a cellular component. Exemplary dyes include, for example, Cy5, Cy5.5, and Cy7, each of which are available from GE Healthcare; VivoTag-680, VivoTag-S680, VivoTag-S750, each of which are available from VisEn Medical; AlexaFluor660, AlexaFluor680, AlexaFluor700, AlexaFluor750, and Alexa Fluor790, each of which are available from Invitrogen; Dy677, Dy676, Dy682, Dy752, Dy780, each of which are available from Dyonics; DyLight547 and DyLight647, each of which are available from Pierce; HiLyte Fluor 647, HiLyte Fluor 680, and HiLyte Fluor 750, each of which are available from AnaSpec; IRDye800CW, IRDye 800RS, and IRDye 700DX, each of which are available from Li-Cor; and ADS780WS, ADS830WS, and ADS832WS, each of which are available from American Dye Source.

As discussed below, NIR labels can be enhanced by metal-enhanced fluorescence (MEF), whereby metallic nanostructures favorably modify the spectral properties of fluorophores and alleviate some of their more classical photophysical constraints. As opposed to other NIR active materials such as carbon nanotubes, the present dyes are water soluble.

Cy5 is a commercially available cyanine dye. Cy5 conjugates are excited maximally at 650 nm and fluoresce maximally at 670 nm. They can be excited to about 98% of maximum with a krypton/argon laser (647 nm line) or to about 63% of maximum with a helium/neon laser (633 nm line). Cy5 can be used with a variety of other fluorophores for multiple labeling due to a wide separation of its emission from that of shorter wavelength-emitting fluorophores. As discussed below, various NIR labels are attached to an analyte capture molecule, such as an antibody, that in turn binds specifically binds to an analyte captured on a bead.

Accordingly, “different metal enhanced fluorescence signals,” are those with emission peaks at different wavelengths. This can be accomplished by using different fluorescent labels, or distinguishing the beads spectroscopically in other ways (e.g. size), as described below.

A “bead”, as used herein, is used in its commonly accepted sense, and refers to a small, often round, piece of solid material. It can be inorganic or polymeric in nature. A bead can have a spherical as well as a nearly spherical, e.g., elliptical, shape. In exemplary embodiments, the beads have cross-sections which are between 0.1 μm and 25 μm, or between 1 μm and 25 μm. The term “bead” is used herein in a very general sense to refer to a microparticle that is essentially inert in the binding aspect of the assay, can be placed in a liquid suspension, and can support multiple macromolecules immobilized thereon. It will generally be on the order of 0.1-50 μm in nominal diameter and have an arcuate surface. For example, the nominal diameter of the bead could be 0.5 μm, 0.75-1 μm, 2 μm, 1-10 μm, 10-20 μm or 20-50 μm. It can be manufactured from various natural and synthetic materials, such as glass, polymer (e.g. glass, silica, Alumina, TiO2, polyethylene, polystyrene, and polymeric latex), ceramic, metal, paramagnetic material, magnetic materials, or carbon. It could be either solid or hollow. It can be externally or internally labeled with a fluorescent dye, or multiple dyes, for purposes of multiplexing, discussed below. The bead could also be referred to as microsphere, nanoparticle or a nanosphere. The term “bead” is used in the present description for convenience.

The microparticles or beads according to the present invention have a certain nominal particle size which ranges from 0.1 μm to 10 μm and preferably from 0.5 μm to 20 μm. The size of the microparticles is essential. The size distribution, e.g. 4 μm or 8 μm, means that the largest percentage, usually at least 80% of all beads are within the given size. Of course there are some beads outside this range since the diameter is distributed statistically. By applying special technique it is, however, possible to make sure that more than 99% of the beads are within the given size.

The term “biotin” also known as “Coenzyme R” or “vitamin H” or “B7” refers to a small molecule with a chemical formula ClOH16N2O3S which is also a water-soluble B vitamin. It is composed of a ureido (tetrahydroimidizalone) ring fused with a tetrahydrothiophene ring. A valeric acid substituent is attached to one of the carbon atoms of the tetrahydrothiophene ring. Biotin is a coenzyme for carboxylase enzymes, involved in the synthesis of fatty acids, isoleucine, and valine, and in gluconeogenesis.

The term “avidin” refers to various forms of avidin, including a compound that is or derives from a 53000 dalton tetrameric protein originally purified from the bacterium Streptomyces avidin, or an egg-white protein, which binds tightly to a small molecule, biotin. Examples include, recombinant streptavidin and derivatives of streptavidin retaining biotin-binding regions. Further examples are given in “Modified avidin and streptavidin molecules and use thereof,” EP 0871658 B1.

The term “spectroscopic” or “spectroscopically” refers to a method to study a sample based on the interaction between matter and radiated energy. A light is applied to a sample area and effect of the light on the sample area is determined. This may include analysis of the reflected or refracted light, or the effect of the light on the sample area, which varies depending on the state of the sample. Spectroscopic methods may be distinguished from chemical or biological methods in which modulation of light does not play a role.

### Overview

The present invention relates to a plasmonic bead based flow cytometric immunoassay having high sensitivity, owing to strong NIR-FE on the gold coated beads. Using a two-step seeding-and-growth approach, one produces gold nano-islands on glass beads. By way of example, these were of two different sizes (8 μm and 4 μm). Through flow cytometry measurement, the inventors then observed up to ˜110 fold enhancement of near-infrared Cy5 fluorophores at ˜650-670 nm emission for Cy5-avidin coated on the gold plasmonic beads compared to those on glass beads. Flow cytometric detection of IL-6, an important human cytokine, on plasmonic beads reached a low limit of detection several orders lower than on glass bead.

Further, there is described a multiplexing scheme for differentiating plasmonic beads by bead size and fluorescence tagging with two visible fluorophores (Alexa fluor 488 dye and Cy3 dye) with non-overlapping emission spectra. A 6-plexed gold-beads system was developed for simultaneous detection of cytokines and a cancer biomarker with sub-pg/ml detection sensitivity and high selectivity, useful for quantification of low biomarker concentration in complicated biological medium.

The plasmonic bead assay has great potential for research and clinical usage for biomarker development and early stage disease (e.g., cancer, autoimmune disease, cardiovascular disease, infectious disease, etc.) detection and treatment.

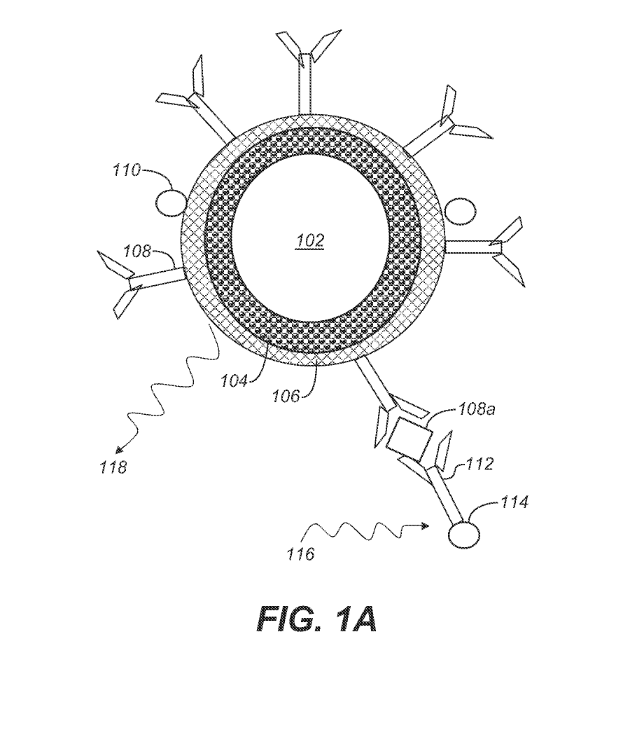

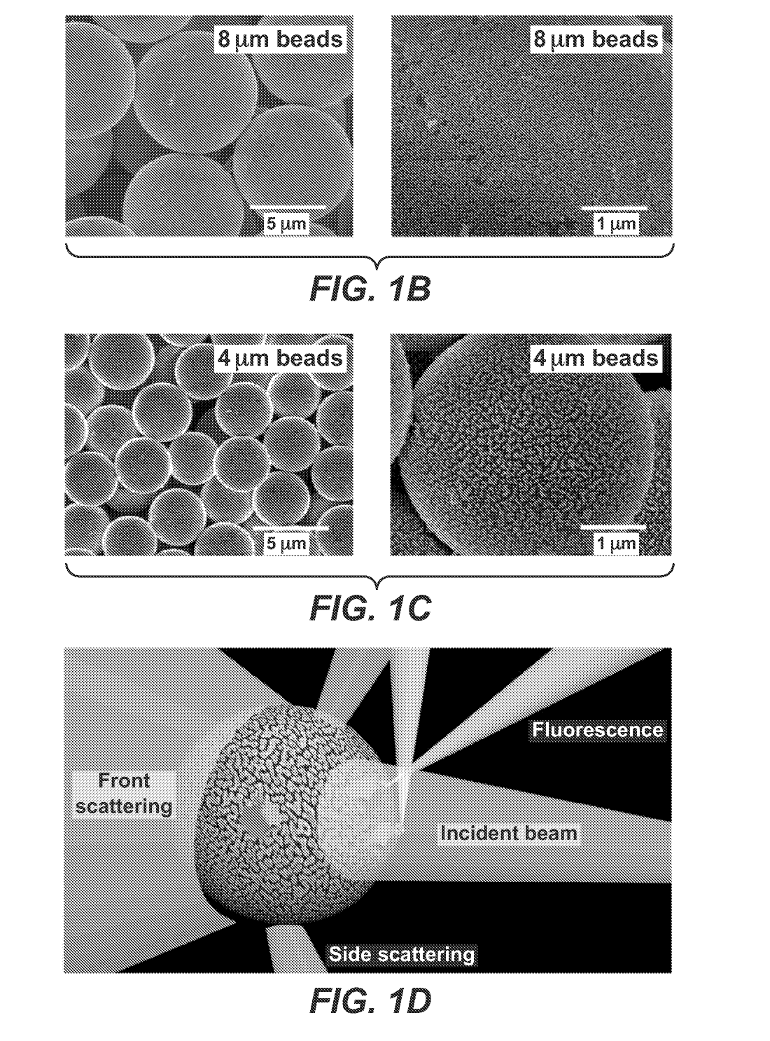

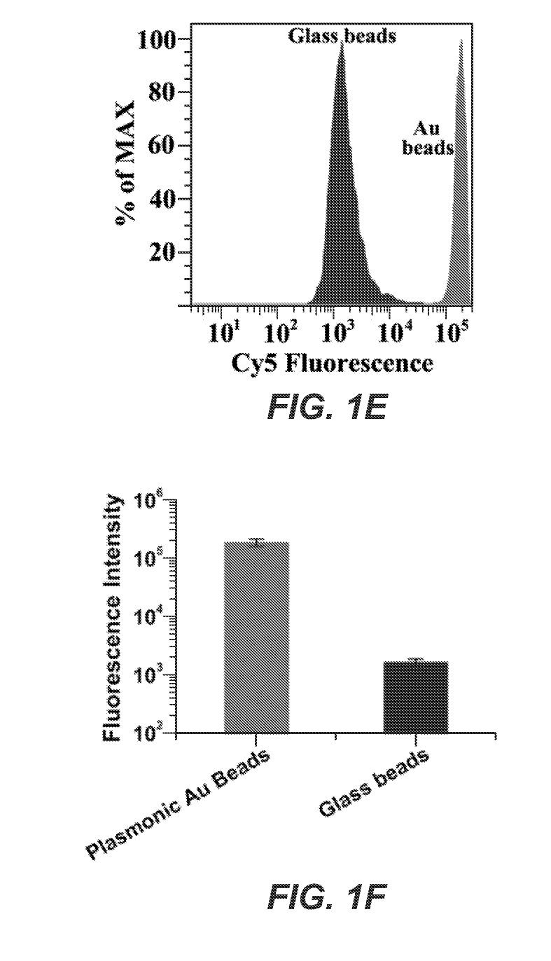

An overview of an embodiment of the present invention is shown in FIG. 1A. Referring now to FIG. 1A, a microparticle 102 such as a glass bead or similar microparticle of a size suitable for use in a suspension or fluidic apparatus and that is essentially inert is shown as the core of the structure. Microparticle 102 is coated with a complete or partially complete nano-structured, plasmonically active metal film 104 applied to and fixed on the microparticle 102. An additional layer (not shown) may be used to provide improved adhesion between the metal film and the microparticle, and in order to provide adhesion between the bead and a second outer layer 106, which is designed for immobilization of recognition molecules 108. For example, the coating layer may be designed to react with pendant silica reactive groups in the bead surface and provide a reactive or adhesive surface for an outer coating of avidin 106. The avidin provides a high affinity coupling for biotinylated recognition molecules 108, shown here as a population of antibodies bound to the outer surface of the microparticle. The recognition molecules will be homogeneous and all recognize the same analyte. As an example, a dense lawn of antibodies, or a monolayer of antibodies may be attached to a single bead. In use, the above-described structure comprising elements 102, 104, 106 and 108 is left intact; that is, the elements are permanently attached to each other and do not dissociate during fluid handling.

Other recognition molecules 108 may be used for other analytes. For example, lectins may be used to detect carbohydrate analytes, or oligonucleotides can be used to detect complementary nucleic acids including DNA and RNA. Receptors can be used to detect ligands for said receptors, such as a T cell receptor for use in detecting T cells in a sample. In use the microsphere structure having a population of recognition antibodies will bind to an analyte 108a, if present in a sample. The assay materials are designed so that a complex of microparticle and analyte bound thereto will exhibit fluorescence when excited by the proper light source shown at 116. To avoid non-specific fluorescence or the need to label the analyte, a secondary antibody 112, bearing a fluorescent label 114 is used. The secondary antibody binds to and detects the antigen 108a on the antibody 108 immobilized on the microparticle. The secondary antibody 112 is labeled with a fluorescent label 114. The fluorescence from the label 114 causes metal-enhanced fluorescence due to the plasmonically active coating 104.

Due to energy coupling between the label 114 and the metal layer 104, the fluorescence that is emitted, shown at 118, is enhanced many fold.

The present assay is designed to be multiplexed, where a number of different analytes can be detected and distinguished in the same sample during the same experiment. This is done, firstly, by providing a population of beads (e.g. 1,000-1,000,000 beads) divided into a subpopulation for each analyte. Each subpopulation can be distinguished from the others by a combination of (i) different recognition molecules 108, (ii) different optical signatures provided by the microsphere size and the metal coating, and/or, optionally, (iii) different labels 110 are attached on or in the beads to distinguish one subpopulation from another. Due to fluorescence emissions at different wavelengths, different analytes on beads can be distinguished, and even separated by flow cytometry and FACS. Label 110 is associated with a bead even in the absence of binding of analyte 108a.

### Assay Sensitivity

Sensitivity is the vital index of low concentration immunoassays, e.g. protein immunoassays. The basic rationale for signal magnification for protein measurement includes: concentrating the analyte to a certain area for analysis (capture analyte by capture antibody, immunoprecipitation, electrophoresis, etc.); reducing non-specific binding of non-targeting proteins which will result in final signal reporting; sensitive signal reporting system (radiolabel for RIA, enzyme for ELISA, fluorophore for bead based immunoassay, etc.). Existing bead based immunoassays such as the Luminex xMAP utilize fluorophores emitting in the visible region for signal reporting (e.g. R-Phycoerythrin) and red dyes for bead coding, providing 3-4 orders of dynamic range and a 1-10 pg/ml level of sensitivity for cytokine measurement, which is similar compared to ELISA. This sensitivity is due to its ability to concentrate cytokines on each bead through immobilization by specific capture antibody on the bead¹³. However, the sensitivity of such an assay is partly limited by fluorescence signal decreasing to a level of the high background due to autofluorescence at the same visible spectrum region.

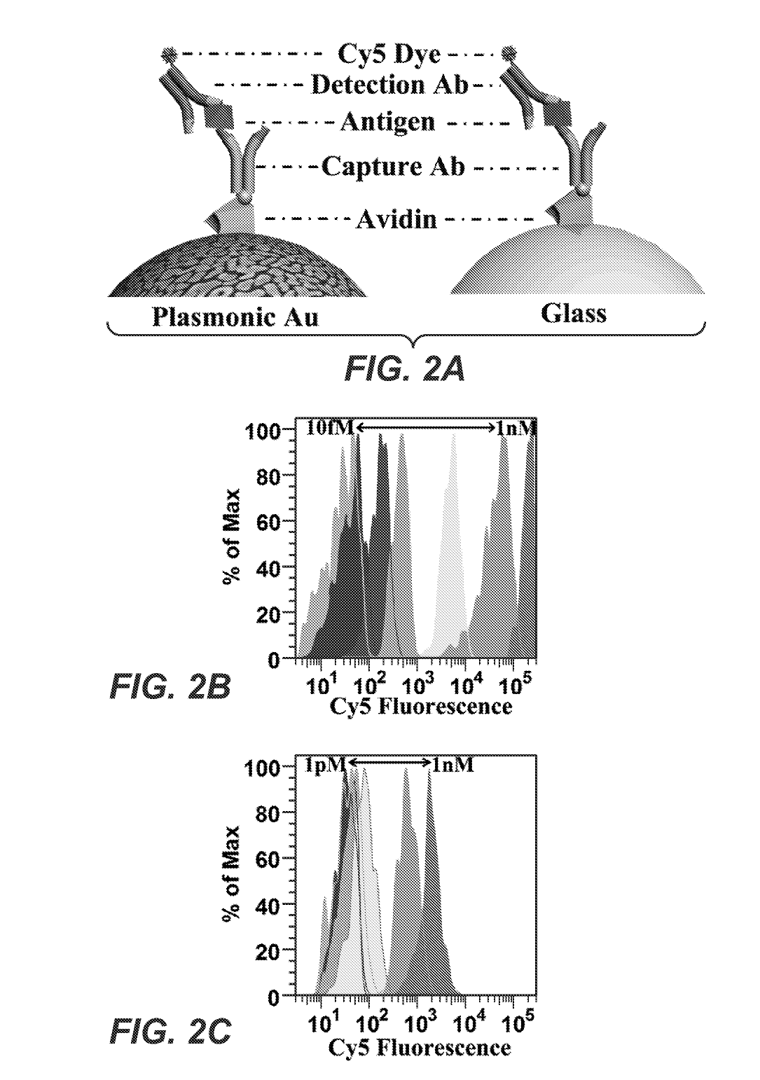

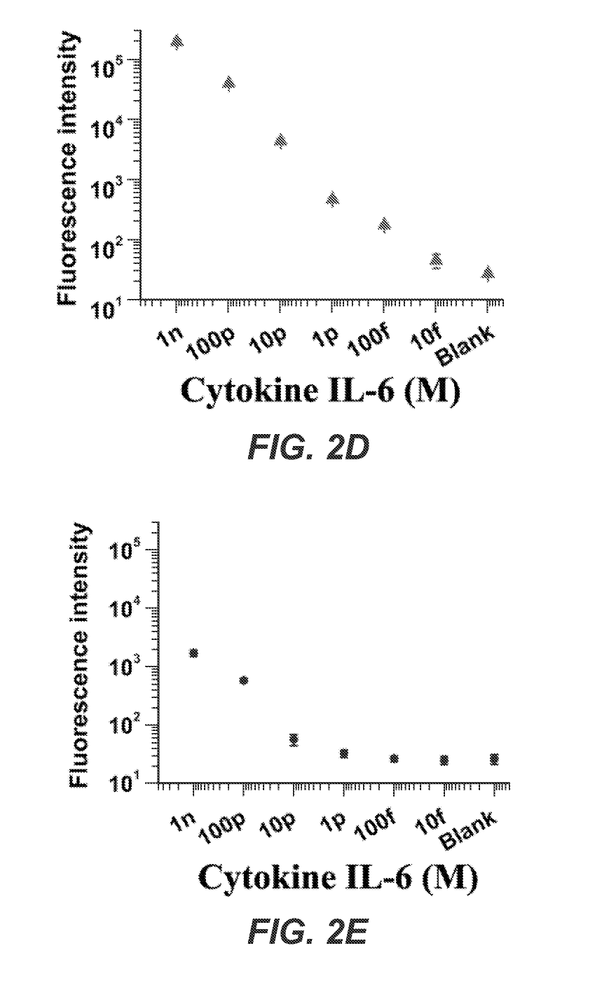

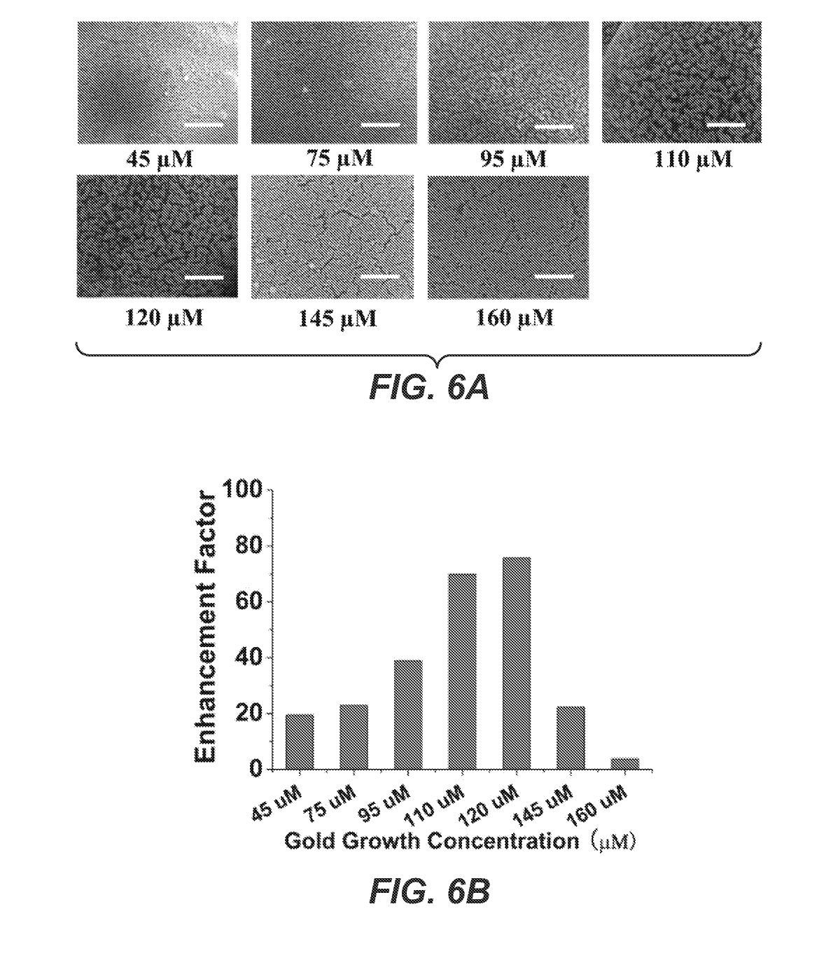

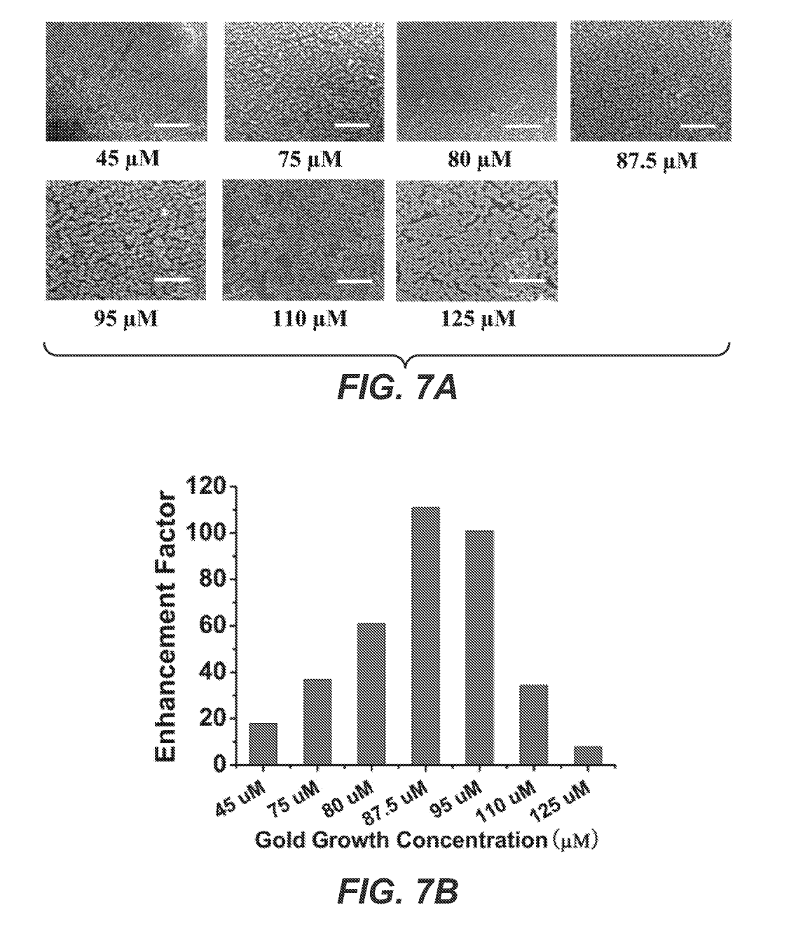

In the present invention, red and near-infrared fluorescence enhancement NIR-FE can be achieved through coating of rationally designed (i.e. having predetermined island sizes and distances [“nanogaps”] between islands in the nm range) Au nanostructures on glass beads (FIG. 6A-6B and FIG. 7A-7B). It was not initially obvious that Au coatings on beads could afford NIR-FE, despite our own observation of NIR-FE of Au coatings on planar substrates. Au coating layer growth on curved bead surfaces differed from growth on planar substrates, leading to a different Au coating morphology. As a result, NIR-FE on beads is enhanced by ˜100 and 50 times for Cy5 and IRDye 800, respectively, which differed from ˜40 times and 100 times for Cy5 and IRDye 800, respectively, on planar substrates. NIR-FE is highly sensitive to Au coating morphology. As described in more detail below, NIR-FE was due to nanogaps in the gold nano-island pattern on the sphere for enhancing the electric fields of local excitation of the fluorophores; and resonance coupling between the emission dipole of NIR fluorophores and plasmon modes in the metal nanostructures (controlled by the size of Au nano-islands) decreasing the radiative lifetime and thus increasing the fluorescence quantum yield²². By utilizing 80-110 fold fluorescence enhancement on plasmonic gold beads, we observed up to 7 orders of dynamic range in protein detection with a low limit of detection (LOD) down to 1 fM (FIG. 2A-2E). On glass beads, ˜1 pM LOD was realized due to fluorescence signal similar to background noise at a biomarker concentration below the 1 pM level. On Au beads, NIR-FE enhances signals below ˜1 pM well above the background which is not enhanced by the Au coating, affording LOD down to fM.

### Biomarkers

Detection of a panel of biomarkers, rather than single-analyte measurements, is likely to improve the sensitivity and specificity in cancer detection, staging and monitoring²⁶⁻²⁸. Cytokines are small proteins secreted by cells and are involved in many diseases including cancer, HIV, Alzheimer's disease and autoimmune diseases¹¹. Simultaneous detection of clinically used low-specificity cancer biomarkers and additional inflammatory cytokines and chemokines could lead to protein profiling of various cancer types, allowing for cancer diagnosis at an early stage^{29,30}.

We demonstrated a multiplexed plasmonic bead system for detecting a panel of cytokines and a cancer protein biomarker (CA-125, Cytokine IL-6, IL-1 beta, IFN-gamma and VEGF) with sub-pg/ml sensitivity, which is 1-2 orders better than existing bead based immunoassay technology, shown in Table 1, below:

TABLE 1
Detection limit of the 5 biomarkers by
multiplexed sensing on plasmonic bead

| | | | Molecular Weight | Detection Limit |
|---|---|---|---|---|
| Biomarker | Detection Limit | | (kD) | (mass concentration) |
| CA-125 | 0.12 | U/mL | | |
| IL-6 | 1.7 | fM | 20.3 | 0.03 pg/mL |
| IFN-gamma | 20.7 | fM | 16.9 | 0.34 pg/mL |
| VEGF | 158 | fM | 19.2 | 3 pg/mL |
| IL-1 beta | 20.8 | fM | 18 | 0.37 pg/mL |

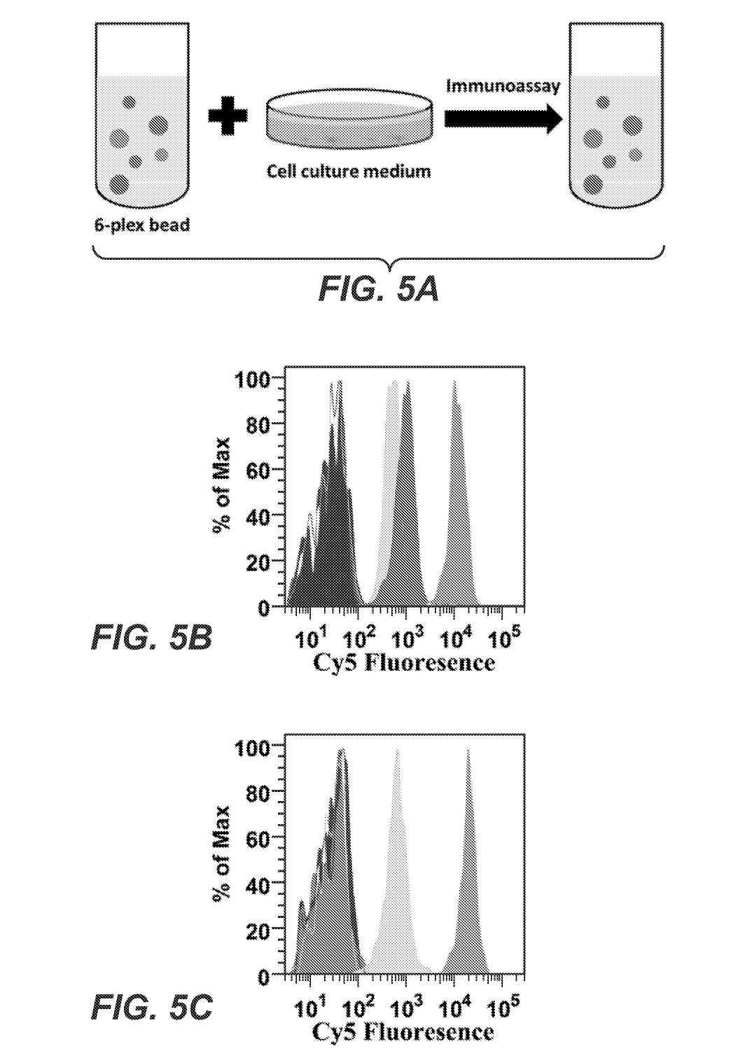

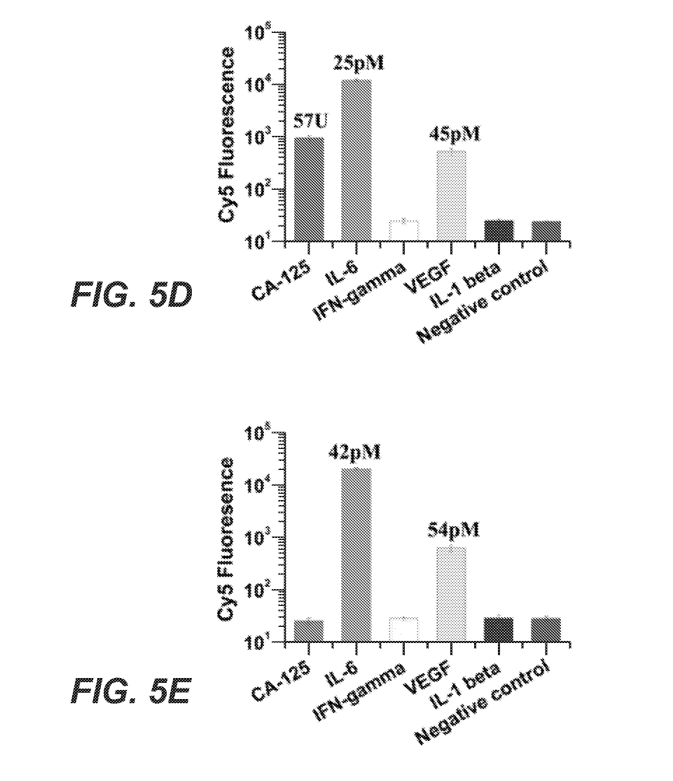

The above biomarkers were detected using antibodies attached to beads as described above and specific for the biomarker of interest. The wide dynamic range of our assay enables bead based quantification of a panel of biomarkers with a wide concentration distribution from sub-pg/ml to tens of ng/ml. VEGF is a potent stimulating factor for angiogenesis and vascular permeability and plays important roles in cancer development³¹. IL-1β, IL-6, and IFN-gamma are hallmark pro-inflammatory cytokines involved in tumor formation and growth³². Specifically, cytokine IL-6 were found to be provocative of tumor formation³² while IFN-gamma was shown to be suppressive of tumor growth³³. Our system detected CA-125 at tens of U/ml and IL-6, VEGF at tens of pM in OVCAR3 cell culture medium, IL-6 and VEGF at tens of pM in SKOV3 cell culture medium but no IL-1 beta or IFN-gamma (FIG. 5C), which is reflective of the roles played by these biomarkers, demonstrating the usefulness of the system for both cancer detection at an early stage and monitoring of cancer treatment.

### Multiplexing

Perhaps one of the most important features of the bead based immunoassay is its capability for multiplexing, which has been done through two ways previously: utilizing multiple sizes of beads for quantification of different analytes²³ and tagging different fluorophores onto beads^{24,25}. Here, we combine the two approaches to afford a single 3-dimensional bead system, enhancing the multiplexing capability of bead based immunoassays. A >4 dimensional plasmonic bead multiplex system can be constructed through selection of different sizes (front scattering), types of bead substrate (side scattering), and fluorophore tagging of plasmonic beads.

### Flow Detection

The present methods utilize plasmonic “beads” that are treated to carry recognition molecules and that are placed into a fluid medium. They may be flowed in such medium as part of the assay process, in which beads are moved into light excitation and light detection devices, as well as other fluidic channels, valves etc. The flow detection may also include flow separation based on the light detection signal. This is referred to as an embodiment of flow cytometry. The term “flow cytometry” refers to the generally accepted use of the term in reference to a device and method in which a beam of light (usually laser light) of a single wavelength is directed onto a hydrodynamically focused stream of liquid. A number of detectors are aimed at the point where the stream passes through the light beam: one in line with the light beam (Forward Scatter or FSC) and several perpendicular to it (Side Scatter or SSC) and one or more fluorescence detectors. Each suspended particle passing through the beam scatters the ray, and fluorescent chemicals found in the particle or attached to the particle may be excited into emitting light at a longer wavelength than the light source. This combination of scattered and fluorescent light is picked up by the detectors, and, by analyzing fluctuations in brightness at each detector (one for each fluorescent emission peak), it is possible to derive various types of information about the physical and chemical structure of each individual particle. Modern flow cytometers are able to analyze several thousand particles every second, in “real time,” and can actively separate and isolate particles having specified properties. Flow cytometry is further described, e.g. in Coulter et al. U.S. Pat. No. 3,710,933, entitled “Multisensor particle sorter,” Burr et al. U.S. Pat. No. 6,079,836, “Flow cytometer droplet break-off location adjustment mechanism,” etc.

In addition, individual beads may be analyzed by a microscope visualizing and measuring fluorescence from a plurality of beads, where individuals may be counted and analyzed by a computer. See, e.g. U.S. Pat. No. 6,159,749, “Detecting substance in sample by mixing antibody coated bead and labeling reagent, inserting beads, sample and labeling reagent into wells, transferring to second well and forming complex, detect labeled complexes,” and U.S. Pat. No. 7,268,861, “Near infrared chemical imaging microscope.”

### Beads

A variety of flow cytometry beads may be used in the present methods, provided that they can be coated with a plasmonically active surface, preferably gold nano-islands. The beads may also be themselves provided with individual labels or colors, e.g. in the Luminex bead system. Beads may be glass, i.e. made from borosilicate glass or specially selected soda lime glass. Beads may also be polystyrene, melamine, PMMA, polylactide, dextran, silica, alumina, or magnetic.

### Materials and Methods Used in Examples

### Materials

1, 4, 8, 10 micron glass beads were purchased from Fiber Optic Center Inc. 2 micron polystyrene bead, [3-(2-aminoethylamino)propyl]trimethoxysilane, chloroauric acid trihydrate, hydroxylamine HCl, sodium borohydride, Cysteamine, Avidin were purchased from Sigma-Aldrich. Ammonium Hydroxide (30% ammonia) and Hyclone fetal bovine serum were purchased from Fisher Chemicals. Purified cytokine antigen standards for VEGF, IL-1β, IL-6 and IFN-γ were purchased from R&D systems. Purified CA-125 antigen was purchased from Fitzgerald industries. Sandwich Antibody pairs for IL-1β, IL-6, and IFN-γ were purchased from R&D systems. Sandwich antibody pairs for VEGF were purchased from Peprotech, inc. Sandwich antibody pair for CA-125 was purchased from Fitzgerald industries. Cy5-NHS and Cy3-NHS ester was purchased from GE-healthcare. Alexa fluor 488-NHS ester was purchased from Invitrogen Life Technologies Corporation. 6-armed poly(ethylene glycol)-amine was purchased from SunBio. Sulfo-SMCC, Biotin-NHS ester was purchased from Thermo Scientific.

### Synthesis of Plasmonic Gold Nano-Island Coated Glass Beads

800 mg 8 micron glass beads were dispersed in 10 mL ethanol with 10% [3-(2-aminoethylamino)propyl]trimethoxysilane and stirred overnight, resulting in amine modified glass beads. The beads were transferred to water solution through centrifuge at 1000 rcf, remove supernatant and resuspend in 50 mL water for three times, resulting in a glass bead solution at 16 mg/ml. 2.5 mL of the bulk solution was diluted with 7.5 mL water, and 100 μL 0.1 M HAuCl₄ and 15 μL fresh Ammonium hydroxide was added, the solution was stirred for 20 min, and washed 3 times by centrifuge at 150 rcf and resuspend in water. The glass bead with Au cluster (1.6 mg/ml, 10 mL) was diluted to 1.6 mg/ml and 24 μL freshly prepared NaBH₄ (0.1 M) was added into the solution and keep stirring for 10 min. The bead with reduced gold seed was washed again by centrifuge at 3000 rcf and resuspend in water for 3 times. Bead with Au seed was diluted to 0.8 mg/ml at 8 mL, same amount of HAuCl4 and Hydroxylamine was added to the solution, resulting in the final HAuCl₄ concentration vary from 50 μM to 150 μM, the solution was stirred for 10 min to allow gold growth on Au seed on glass beads, the beads were finally washed by centrifuge at 3000 g and resuspend in water. Concentration of gold salt, sodium borohydride and stirring speed are optimized for bead coating. Higher stirring speeds (around 800 rpm) produced better coatings.

### Single-Plex Immunoassay on Plasmonic Bead

100,000 plasmonic beads (counted by flow cytometry) were coated with avidin by soaking the beads in 1 μM avidin/PBS solution at 4 C overnight, following by washing with PBS solution. The avidin coated plasmonic bead was incubated with 10 nM biotinylated mouse anti IL-6 capture antibody for 3 h, washed 2 times with PBST and 1 time with PBS. The bead was later blocked with biotinylated branched PEG and 20% FBS in PBS. 5000 IL-6 capture antibody labeled beads was distributed into each well, 100 μL serial dilution of IL-6 antigen from 1 nM to 10 fM in PBS solution with 20% FBS was added to each well, incubate at room temperature for 1 h and washed 2 times with PBST and 1 time with PBS. 100 μL 10 nM Cy5 labeled mouse anti IL-6 detection antibody in PBS with 20% FBS was added to each well, incubate at room temperature for 1 h in dark, washed 2 times with PBST and 1 time with PBS.

### Construction of 3-Dimensional Plasmonic Bead System for Multiplexed Protein Sensing

Plasmonic beads were mixed with 10 μM fluorophore-NHS ester in DMSO solution and incubated at room temperature in the dark for 2 h, and during incubation the fluorophore was linked with free amine groups between the gaps of the gold islands on the beads. The fluorophore labeled bead was then coated with avidin by soaking the beads in 1 μM avidin/PBS solution at 4 C overnight, following by washing with PBS solution. The avidin coated plasmonic bead was incubated with 10 nM biotinylated capture antibody for 3 h, and blocked with biotinylated branched PEG and 20% FBS in PBS. 8 micron sphere with CA-125 capture antibody, Alexa 488 coded 8 micron sphere with IFN-gamma capture antibody, Cy3 coded 8 micron sphere with IL-6 capture antibody, 4 micron sphere with VEGF capture antibody, Alexa 488 coded 4 micron sphere with IL-1 beta capture anti body, Cy3 coded 4 micron sphere with PEG for negative control were constructed by following the procedure above and were mixed together to form the 3-dimensional plasmonic bead system.

### OVCAR-3 and SKOV-3 Cell Culture

SKOV-3 cells were cultured in McCoy's 5A Medium with L-glutamine, and OVCAR-3 cells were cultured in RPMI Medium 1640 with L-glutamine. Both culture media were supplemented with 10% fetal bovine serum, 100 IU·mL−1 penicillin and 100 μg/mL streptomycin. Cells were maintained in a 37° C. incubator with 5% CO2 for 48 hrs at 50-60% confluency, before the supernatant was sampled for microarray sensing. As a control, fresh cell medium without cells growing was also used for sensing.

### EXAMPLES

### Example 1

### Producing Plasmonic Gold Beads

Plasmonic gold coated silica beads were prepared through a two-step seeding-and-growth approach¹⁸. First, glass beads (8 μm or 4 μm in diameter) were modified with amine groups through reaction with [3-(2-aminoethylamino)propyl]trimethoxysilane (AEPTS). The amine modified glass beads were introduced to a HAuCl₄ solution followed by adding ammonium hydroxide (see methods), resulting in [Au(OH)ₓ(NH3)_{y}Cl_{z}]ₘⁿ⁺ clusters attaching to amine modified glass beads. The clusters were then reduced to gold nanoparticles (gold seeds) by sodium borohydride. Growth of gold on the seed particles was performed by introducing the gold seeded glass beads into a solution composed of HAuCl₄ and NH₂OH for reducing Au(III) selectively on the gold seeds on the bead by NH₂OH. The color of the resulting bead solution is blue-purple, correlating with red and near-infrared plasmonic absorption of the bead. Scanning electron microscopy (SEM) revealed that the gold coating on the glass bead contained tortuous gold islands uniformly covering the glass beads (FIG. 1C and FIG. 1D). We found that successful synthesis of plasmonic Au beads relied on amine modification of glass beads for gold seeding on glass beads, and the HAuCl₄ concentration in the growth step was important to the morphology of uniform gold nano-island coating.

### Example 2

### Fluorescence Enhancement of Near-Infrared Fluorophore on Plasmonic Beads

We investigated the fluorescence enhancement of gold coated beads with various coating morphology by absorbing Cy5-avidin onto the beads via non-specific binding. Cy5-avidin was also absorbed on glass beads for comparison. The Cy5 fluorescence intensity (peak˜670 nm) on plasmonic beads and on glass beads were quantified by flow cytometry (FIG. 1D). A vial containing 100,000 Cy5-avidin coated plasmonic beads was placed in a flow cytometer, with each individual bead passed through a micro fluidic channel for detection of front scattering and side scattering using an incident 488 nm laser. Simultaneously, Cy5 fluorophores on the bead were excited with a 640 nm laser with its fluorescence emission recorded.

We observed that at low growth concentration of HAuCl₄ (<50 μM), discrete gold nanoparticles were formed on glass beads, giving low NIR-FE effects (FIG. 6A-6B and FIG. 7A-7B). As the growth concentration increased, the gold nanoparticles began to transform into larger gold-islands and the gaps between gold-islands decreased, accompanied by an increase in Cy5 fluorescence enhancement quantified by flow cytometry. At very high growth concentrations, the gold-islands began to coalesce to form continuous gold films, resulting in a significant drop in Cy5 fluorescence due to quenching (FIG. 6A-6B and FIG. 7A-7B). We found that optimized plasmonic beads contained gold islands at 100 nm scale (e.g. 50-200 nm) with gaps between gold-islands at 10-30 nm range. The Au beads afforded ˜110 times higher fluorescence on plasmonic bead over on glass bead (FIG. 1E and FIG. 1F).

### Example 3

### Plasmonic Gold Beads for Single Cytokine Detection

The 8 micron gold plasmonic beads were coated with avidin and then biotinylated capture antibody specific to the human cytokine IL-6. After blocking with biotinylated branched polyethylene glycol (PEG) and fetal bovine serum (FBS), the beads were distributed into multiple vials with ˜5000 beads in each vial. Serially diluted human cytokine IL-6 solutions from 1 nM to 10 fM plus a blank control were added to each vial of bead modified with anti-human IL-6 capture antibody. After equilibration, washing and incubation with fluorophore Cy5 labeled anti human IL-6 detection antibody (FIG. 2A), ˜1000 beads in each vial were counted by flow cytometry with Cy5 fluorescence intensity measured for each concentration of IL-6 (FIG. 2B-2C). We observed 7 orders of magnitude dynamic range in IL-6 detection, spanning from 1 nM IL-6 down to 10 fM using 100 μL solution. The calculated low limit of detection of human IL-6 by the plasmonic bead assay is ˜2 fM, which is defined by fitting background signal plus two standard deviations into the IL-6 calibration curve (FIG. 2D-2E). This sensitivity corresponded to detecting ˜10 fluorophores per plasmonic bead, which was two orders more sensitive than the commercial Luminex xMap technology¹⁹.

The same immunoassay was also constructed on glass beads (FIG. 2A), and a ˜500 fM LOD was reached without any NIR-FE effect on the glass beads. This sensitivity also matched the commercial Luminex technology (˜10 pg/ml) (FIG. 2B-2C and FIG. 2D-2E). With 4 μm plasmonic gold beads, we observed a lower NIR-FE by ˜80 times, but the LOD for human IL-6 quantification was similar to that obtained with 8 μm Au beads (data not shown).

### Example 4

### Multiplexed Plasmonic Beads for Protein Profiling

Multiplexed bead based immunoassays have been made possible through quantification of different analytes on beads with different sizes²⁰, or with schemes utilizing two fluorophores for tagging beads with different ratios of the fluorophores. Here, we used similar approaches for plasmonic gold beads utilizing both bead size and fluorescent dye tagging of the beads. A prototype of this system is demonstrated in this work, where 4 and 8 micron plasmonic beads were tagged with Cy3 and Alexa Fluor 488. We constructed a system for multiplexed quantification of human cytokines and human ovarian cancer biomarker CA-125.

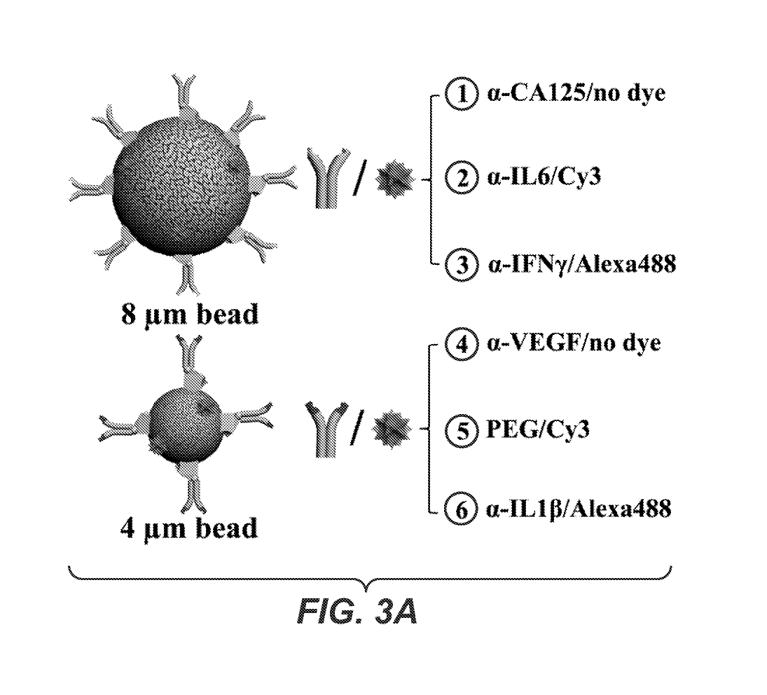

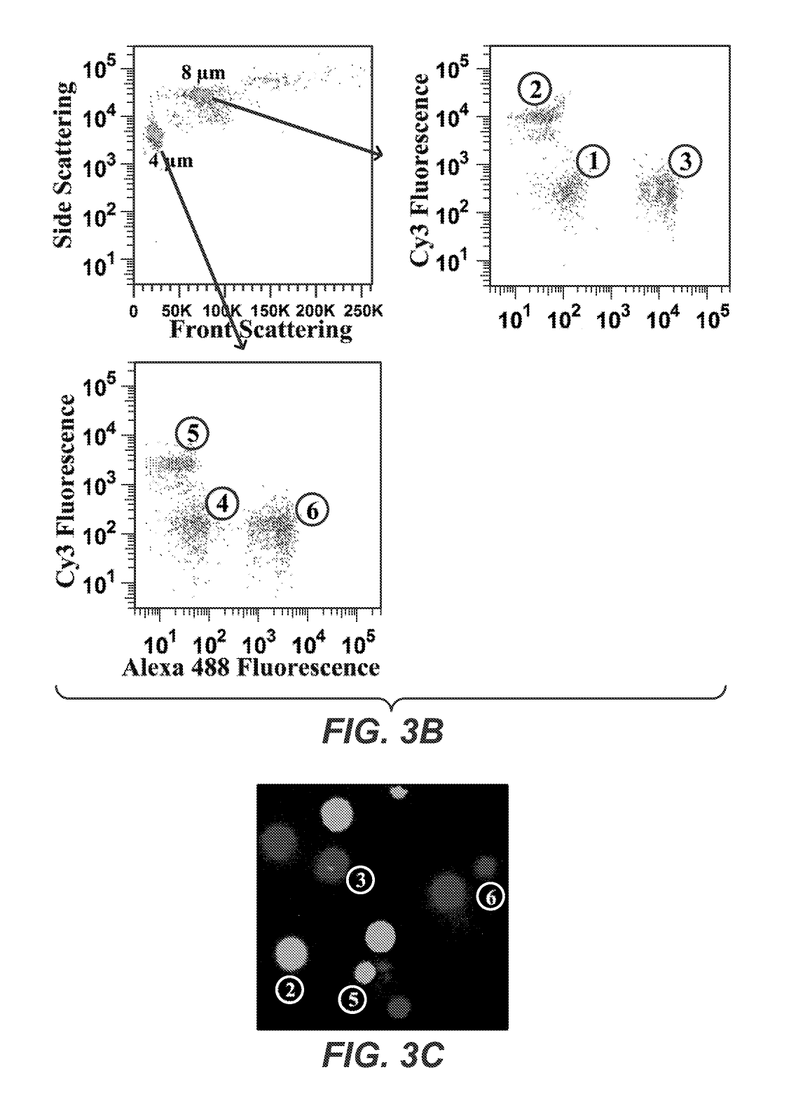

Fluorophore tagging of the plasmonic beads was achieved through reaction of NHS functionalized dye (Cy3 or Alexa 488) with amine groups on the glass surface between the gold-island gaps. Each type of bead was fabricated separately and was later mixed together to form the multiplexed protein sensing system. To achieve this, 6 types of plasmonic beads were constructed: 8 micron sphere with CA-125 capture antibody without fluorescence tagging; Alexa 488 coded 8 micron sphere with IFN-gamma capture antibody; Cy3 coded 8 micron sphere with IL-6 capture antibody; and 4 micron sphere with VEGF capture antibody without fluorescence tagging; Alexa 488 coded 4 micron sphere with IL-1 beta capture antibody; and Cy3 coded 4 micron sphere with PEG for negative control (FIG. 3A).

A mixture of 30,000 6-plexed beads (5,000 of each type) was used for protein detection and 6,000 of those were recorded through flow cytometry measurement. Light scattering plot (side scatter versus front scatter) clearly revealed two different regions of beads, corresponding to 4 micron sphere and 8 micron spheres respectively (FIG. 3B). The 4 micron spheres and 8 micron spheres are further differentiated into 3 sub regions based on the Cy3 and Alexa 488 fluorescence intensities, which is also revealed by confocal fluorescence imaging (FIG. 3B-3C). This result confirmed the feasibility of plasmonic bead multiplexing, as the size of beads can be determined by front scattering, and the fluorescence tagging of the bead can be detected by flow cytometry which allows us to determine the capture antibodies pre-immobilized on the bead.

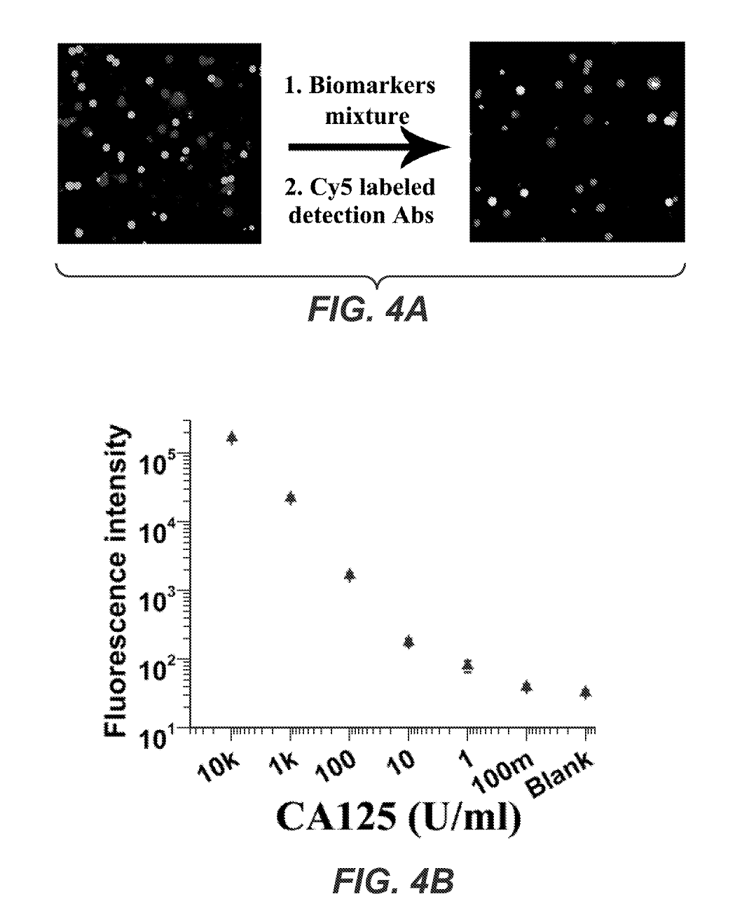

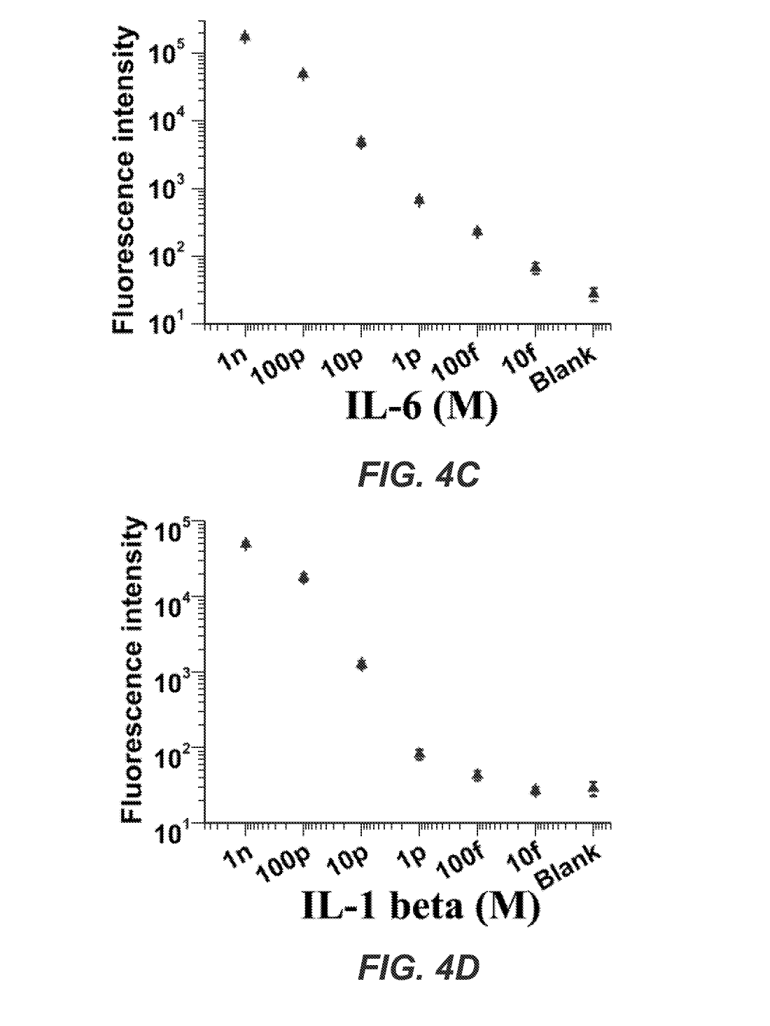

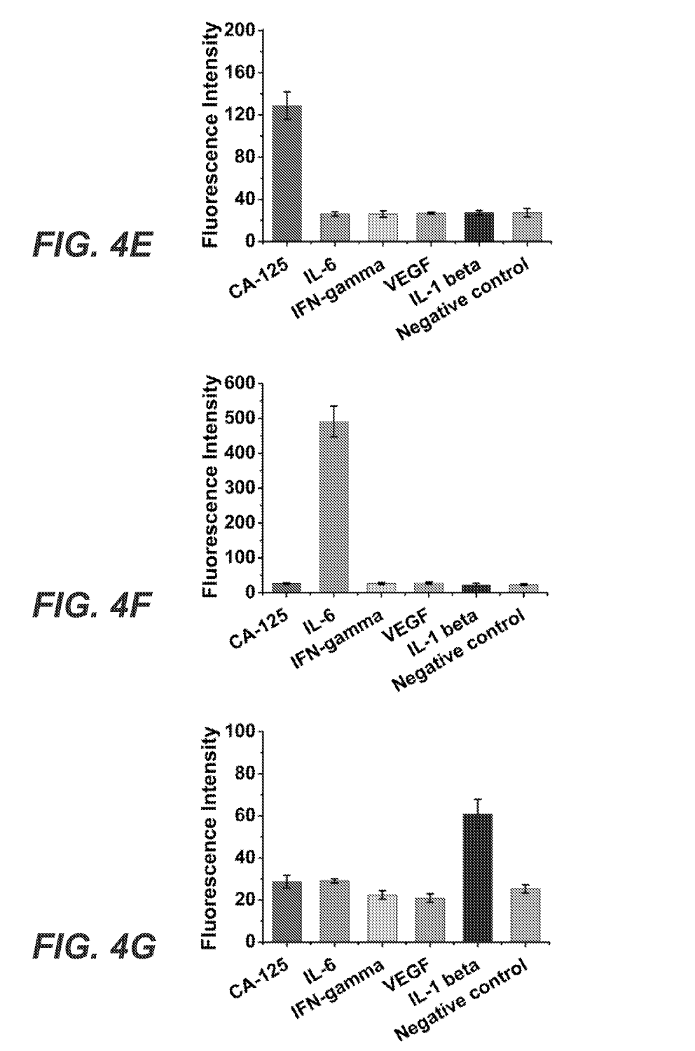

The dynamic range and sensitivity of this multiplexed system was assessed through calibrating serial dilutions of CA-125, IL-6, IFN-gamma, VEGF and IL-1 beta mixture in the 1 nM-1 fM range for each protein. After probing with the mixture of biomarkers, a mixture of Cy5 conjugated detection antibody, each specific to one biomarker, was introduced to the plasmonic bead solution for labeling the biomarkers on each bead with Cy5 dye (FIG. 4A). 5-7 orders of dynamic range was achieved for each analyte with down to 100 fM to 1 fM sensitivity (FIG. 4B-4D), determined by fitting blank plus two standard deviations into the calibration curve. Sub-pg/ml cytokine sensitivity was achieved on this system and CA-125 was found detectable at 0.12 U/mL (Table 1).

The specificity of multiplexed protein detection was confirmed through detection of individual types of proteins by the 6-plex plasmonic bead system. Human cytokine IL-6, IFN-gamma, IL-1 beta, VEGF and ovarian cancer biomarker CA-125 was each spiked into a mixture of 6-plex plasmonic bead solution and detected with a mixture of Cy5 labeled detection antibodies against each analyte. Flow cytometry measurement indicated that only one subset of plasmonic beads corresponding to the spiked protein was labeled with Cy5, demonstrating the selectivity of this system for cytokine quantification at the sub-pg/ml level (FIG. 4E-4G).

### Example 5

### Plasmonic Bead Based Multiplex Protein Biomarker Quantification in Biological Medium

To demonstrate the capability of our multiplexed plasmonic bead system for protein quantification in complex biological samples, we performed cytokine and CA125 detection in cancer cell culture medium through a flow cytometric immunoassay on plasmonic beads. Human ovarian cancer cell lines OVCAR3 and SKOV3 were cultured for 48 h and their culture media were collected for cytokine and CA125 measurements (FIG. 5A). OVCAR3 is an ovarian cancer cell line known to excrete CA125 while SKOV3 is a CA125 negative ovarian cancer cell line²¹. We detected high CA-125, IL-6 and VEGF expression levels in the OVCAR3 culture medium, and IL-6 and VEGF in the SKOV3 culture medium (FIG. 5B-5C). The concentration of each analyte was determined by fitting the Cy5 fluorescence signal to the corresponding calibration curve (FIG. 4B-4D). 25 pM of IL-6, 45 pM of VEGF and 57 U/ml of CA-125 were detected in the OVCAR3 culture medium; while 42 pM IL-6 and 54 pM VEGF was observed the in SKOV3 culture medium (FIG. 5D-5E).

### CONCLUSION

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent or publication pertains as of its date and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material to which is referred.

### REFERENCES

1 Horan, P. K. & Wheeless, L. L., Jr. Quantitative single cell analysis and sorting. Science 198, 149-157 (1977).
2 Krishhan, V. V., Khan, I. H. & Luciw, P. A. Multiplexed microbead immunoassays by flow cytometry for molecular profiling: Basic concepts and proteomics applications. Crit Rev Biotechnol 29, 29-43, doi:10.1080/07388550802688847 (2009).
3 Fulton, R. J., McDade, R. L., Smith, P. L., Kienker, L. J. & Kettman, J. R., Jr. Advanced multiplexed analysis with the FlowMetrix system. Clinical chemistry 43, 1749-1756 (1997).
4 Jani, I. V., Janossy, G., Brown, D. W. & Mandy, F. Multiplexed immunoassays by flow cytometry for diagnosis and surveillance of infectious diseases in resource-poor settings. Lancet Infect Dis 2, 243-250, doi:S1473309902002426 [pii] (2002).
5 Martins, T. B. et al. Determination of cytokine responses using a multiplexed fluorescent microsphere immunoassay. Am J Clin Pathol 118, 346-353 (2002).
6 Siawaya, J. F. D. et al. An Evaluation of Commercial Fluorescent Bead-Based Luminex Cytokine Assays. PloS one 3 (2008).
7 Du, J. et al. Bead-based profiling of tyrosine kinase phosphorylation identifies SRC as a potential target for glioblastoma therapy. Nature biotechnology 27, 77-83, doi:10.1038/nbt.1513 (2009).
8 Lu, J. et al. MicroRNA expression profiles classify human cancers. Nature 435, 834-838, doi:10.1038/nature03702 (2005).
9 Carson, R. T. & Vignali, D. A. Simultaneous quantitation of 15 cytokines using a multiplexed flow cytometric assay. Journal of immunological methods 227, 41-52 (1999).
10 de Jager, W., te Velthuis, H., Prakken, B. J., Kuis, W. & Rijkers, G. T. Simultaneous detection of 15 human cytokines in a single sample of stimulated peripheral blood mononuclear cells. Clinical and diagnostic laboratory immunology 10, 133-139 (2003).
11 Kindt, T. J., Goldsby, R. A., Osborne, B. A. & Kuby, J. Kuby immunology. 6th edn, (W.H. Freeman, 2007).
12 Kleiner, G., Marcuzzi, A., Zanin, V., Monasta, L. & Zauli, G. Cytokine levels in the serum of healthy subjects. Mediators of inflammation 2013, 434010, doi:10.1155/2013/434010 (2013).
13 Chowdhury, F., Williams, A. & Johnson, P. Validation and comparison of two multiplex technologies, Luminex and Mesoscale Discovery, for human cytokine profiling. J Immunol Methods 340, 55-64, doi:S0022-1759(08)00311-6 [pii]10.1016/j.jim.2008.10.002 (2009).
14 Aslan, K. et al. Metal-enhanced fluorescence: an emerging tool in biotechnology. Current opinion in biotechnology 16, 55-62, doi:10.1016/j.copbio.2005.01.001 (2005).
15 Tabakman, S. M. et al. Plasmonic substrates for multiplexed protein microarrays with femtomolar sensitivity and broad dynamic range. Nature communications 2, 466, doi:10.1038/ncomms1477 (2011).
16 Zhang, B. et al. Multiplexed cytokine detection on plasmonic gold substrates with enhanced near-infrared fluorescence. Nano Res 6, 113-120 (2013).
17 Deng, W., Drozdowicz-Tomsia, K., Jin, D. Y. & Goldys, E. M. Enhanced Flow Cytometry-Based Bead Immunoassays Using Metal Nanostructures. Analytical chemistry 81, 7248-7255 (2009).
18 Tabakman, S. M., Chen, Z., Casalongue, H. S., Wang, H. & Dai, H. A new approach to solution-phase gold seeding for SERS substrates. Small 7, 499-505, doi:10.1002/sm11.201001836 (2011).
19 Luminex xMap technology. http://www.millipore.com/bmia/flx4/multiplexing-instruments#tab1=2
20 McHugh, T. M., Miner, R. C., Logan, L. H. & Stites, D. P. Simultaneous detection of antibodies to cytomegalovirus and herpes simplex virus by using flow cytometry and a microsphere-based fluorescence immunoassay. Journal of clinical microbiology 26, 1957-1961 (1988).
21 Boivin, M., Lane, D., Piche, A. & Rancourt, C. CA125 (MUC16) tumor antigen selectively modulates the sensitivity of ovarian cancer cells to genotoxic drug-induced apoptosis. Gynecologic oncology 115, 407-413, doi:10.1016/j.ygyno.2009.08.007 (2009).
22 Tabakman, S. M. et al. Plasmonic substrates for multiplexed protein microarrays with femtomolar sensitivity and broad dynamic range. Nat Commun 2, doi:Artn 466Doi 10.1038/Ncomms1477 (2011).
23 Hartmann, M., Roeraade, J., Stoll, D., Templin, M. F. & Joos, T. O. Protein microarrays for diagnostic assays. Anal Bioanal Chem 393, 1407-1416, doi:10.1007/s00216-008-2379-z (2009).
24 Braeckmans, K., De Smedt, S. C., Leblans, M., Pauwels, R. & Demeester, J. Encoding microcarriers: present and future technologies. Nat Rev Drug Discov 1, 447-456, doi:10.1038/nrd817 (2002).
25 Elshal, M. F. & McCoy, J. P. Multiplex bead array assays: performance evaluation and comparison of sensitivity to ELISA. Methods 38, 317-323, doi:S1046-2023(06)00010-7 [pii]10.1016/j.ymeth.2005.11.010 (2006).
26 Huang, R. P. et al. Cytokine Antibody Arrays in Biomarker Discovery and Validation. Curr Proteomics 9, 55-70 (2012).
27 Hanash, S. M., Pitteri, S. J. & Faca, V. M. Mining the plasma proteome for cancer biomarkers. Nature 452, 571-579, doi:nature06916 [pii]10.1038/nature06916 (2008).
28 Kulasingam, V. & Diamandis, E. P. Strategies for discovering novel cancer biomarkers through utilization of emerging technologies. Nat Clin Pract Oncol 5, 588-599, doi:ncponc1187 [pii]10.1038/ncponc1187 (2008).
29 Zhang, Z. et al. Three biomarkers identified from serum proteomic analysis for the detection of early stage ovarian cancer. Cancer Res 64, 5882-5890, doi:10.1158/0008-5472.CAN-04-074664/16/5882 [pii] (2004).
30 Mor, G. et al. Serum protein markers for early detection of ovarian cancer. Proc Natl Acad Sci USA 102, 7677-7682, doi:0502178102 [pii]10.1073/pnas.0502178102 (2005).
31 Carmeliet, P. Angiogenesis in life, disease and medicine. Nature 438, 932-936, doi:nature04478 [pii]10.1038/nature04478 (2005).
32 Karin, M. Nuclear factor-kappaB in cancer development and progression. Nature 441, 431-436, doi:nature04870 [pii]10.1038/nature04870 (2006).
33 Dranoff, G. Cytokines in cancer pathogenesis and cancer therapy. Nat Rev Cancer 4, 11-22, doi:10.1038/nrc1252nrc1252 [pii] (2004).

1. A method for detection of analytes in a sample, comprising:
(a) contacting the sample with a population of microparticles in suspension, said microparticles comprising a plasmonically active surface providing near-infrared (“NIR”) enhanced fluorescence, said fluorescence optionally provided by gold nano-island covering a portion of said microparticles, said population of microparticles further comprising subpopulations comprising different analyte capture molecules bound to said plasmonically active surface;
(b) allowing said different analyte capture molecules to form complexes with different analytes that may be present in the sample;
(c) labeling complexes formed in step (b) with fluorescent labels;
(d) detecting labeled complexes by irradiating said fluorescent labels and sensing metal enhanced fluorescence from labeled complexes, whereby NIR enhanced fluorescence from said microparticles indicates detection of analytes; and
(e) distinguishing multiple analytes in the sample, if present, by different NIR enhanced fluorescence signals.

2. The method of claim 1 wherein the step of distinguishing multiple analytes is carried out and comprises use of at least one of
(i) different fluorescent labels attached to different captured analytes;
(ii) different microparticle sizes used to detect different analytes; and
(iii) different labels affixed to different microparticles used to detect different analytes.

3. The method of claim 2 wherein the step of distinguishing multiple analytes in the sample comprises measuring said different enhanced NIR fluorescent signals by flow cytometry.

4. The method of claim 1 wherein said subpopulations comprising different analyte capture molecules comprise different sized beads.

5. The method of claim 1 wherein the fluorescent labels are NIR dyes having emissions in the range of 650-800 nm.

6. The method of claim 1 wherein the plasmonically active surface comprises gold nano-islands.

7. The method of claim 6 wherein the plasmonically active surface comprises gold nano-islands coated on beads, said beads further selected from the group consisting of amine-functionalized silica-based, polymer-based and magnetic beads.

8. The method of claim 7 wherein said microparticles comprise an avidin layer on top of the plasmonically active surface.

9. The method of claim 6 wherein said analyte capture molecules are selected from the group consisting of antibodies, antigens, nucleic acids, carbohydrates, and binding peptides.

10. The method of claim 9 wherein said analyte capture molecules are biotinylated antibodies bound to an avidin layer on said microparticles.

11. The method of claim 5 wherein the steps of labeling complexes comprise steps of providing said analyte capture molecules selected from the group consisting of antibodies, antigens, DNA, and peptides, wherein said capture molecules are immobilized on surfaces of microparticles; and providing a fluorescent label in the form of fluorescently labeled antibodies specific to said analytes, whereby a complex is formed between capture molecules, analyte, and fluorescently labeled antibodies or streptavidin.

12. The method of claim 1, wherein the step of distinguishing multiple analytes is done with antibodies having different specificities and labeled with different NIR labels having non-overlapping emission spectra.

13. A product comprising a population of beads wherein each bead comprises, on an outer surface thereof, gold islands separated by gaps of between 5 and 100 nm, and said gold islands have an area between either 1,000 and 2,500 nm², or 25 and 250,000 nm², said population of beads further having coupled thereto analyte capture molecules of at least two different specificities.

14. The product of claim 13 wherein said population of beads comprises at least two different sizes of beads.

15. The product of claim 13 wherein said population of beads comprises beads of silica-based, polymer-based, or magnetic material.

16. The product of claim 13 wherein said population of beads comprises beads of silica-based material modified with an amine functionality coupled to a plasmonically active layer.

17. The product of claim 13 wherein said population of beads comprises beads that are of sizes that differ by at least a factor of 1.5.

18. The product of claim 13 wherein said beads comprise different fluorescent labels.

19. The product of claim 13 wherein said population of beads comprises beads having outer surface gold islands of different sizes as between beads.

20. The product of claim 13 wherein said analyte capture molecules are antibody molecules of at least two different specificities.

21. The product of claim 13 wherein said analyte capture molecules are selected from the group consisting of antibodies, antigens, carbohydrates, nucleic acids and binding peptides.

22. A method of using an immunoassay product as defined in claim 13, comprising the steps of:
(a) forming a mixture of a sample with said population of beads;
(b) allowing capture molecules to bind to analytes in said sample;
(c) separating beads with bound analytes;
(d) forming a complex of bound analytes and fluorescently labeled detection molecules; and
(e) detecting the complex of step (d) by fluorescence, on a bead-by-bead basis, wherein said fluorescence is enhanced NIR fluorescence resulting from interaction between fluorescent labels on the fluorescently labeled detection molecules and the plasmonically active layer.

23. The immunoassay product of claim 22 wherein said detecting bead-by-bead is done by flow cytometry.

24. The immunoassay product of claim 22 wherein said detecting bead-by-bead is done by microscope imaging.

25. The immunoassay product of claim 23 wherein said flow cytometry distinguishes between different analytes by measuring both scattering and fluorescent emission peaks at different wavelengths.

26. The immunoassay product of claim 22 wherein the analyte is an antibody of a human or other species.

27. The immunoassay product of claim 22 wherein the fluorescently labeled detection molecules are antibodies that are labeled with an NIR dye.

28. A method of making a bead-based immunoassay product comprising a population of beads, comprising the steps of:
(a) modifying beads having a material that is one of a silica-based, polymer-based, or magnetic material with an amine functionality
(b) coupling the beads to a plasmonically active layer comprising gold islands separated by gaps of 5 and 100 nm and wherein the islands are between 1,000 and 2,500 nm², or between 25 and 250,000 nm², in area;
(c) applying to the beads a functionality for coupling thereto a population of detection molecules; and
(d) coupling detection molecules of different specificities to the beads as prepared in steps (a)-(c).

29. The method of claim 2, wherein the fluorescent labels are NIR dyes having emissions in the range of 650-800 nm.

30. The method of claim 3, wherein the fluorescent labels are NIR dyes having emissions in the range of 650-800 nm.

31. The method of claim 2, wherein the plasmonically active surface comprises gold nano-islands.

32. The method of claim 2, wherein the step of distinguishing multiple analytes is carried out with antibodies having different specificities and labeled with different NIR labels having non-overlapping emission spectra.

33. The method of claim 3, wherein the plasmonically active surface comprises gold nano-islands.

34. The method of claim 3, wherein the step of distinguishing multiple analytes is carried out with antibodies having different specificities and labeled with different NIR labels having non-overlapping emission spectra.

35. The method of claim 4, wherein the step of distinguishing multiple analytes is carried out with antibodies having different specificities and labeled with different NIR labels having non-overlapping emission spectra.

36. The product of claim 13, wherein said population of beads comprises between three and five different sizes of beads.
